(19) United States
(12) Patent Application Publication
(10) Pub. No.: US 2006/0063980 A1
(43) Pub. Date: Mar. 23, 2006

(54) Mobile phone apparatus for performing sports physiological measurements and generating workout information

(75) Inventors: Yuh-Swu Hwang, Tainan (TW); York-Yih Sun, Tainan (TW)

Correspondence Address: Ladas & Parry, Suite 2100, 5670 Wilshire Boulevard, Los Angeles, CA, 90036-5679, US

(73) Assignee: Yuh-Swu Hwang

(21) Appl. No.: 11/273,764

(22) Filed: Nov. 14, 2005

(30) Foreign Application Priority Data
Apr. 22, 2004 (TW) 093111192

(51) Int. Cl. G06Q 10/00; G21C 17/00; A61B 5/00; G06Q 50/00; G06F 11/30

(52) U.S. Cl. 600/300; 705/2; 702/183

(57) ABSTRACT

A mobile phone apparatus for performing sports physiological measurements and generating target workout information includes a motion detector, a physiological parameter detector, a portable housing, and a processing module. The motion detector detects motion of a user performing exercise, the physiological parameter detector detects physiological parameters of the user, the portable housing houses the processing module, and the processing module is coupled to the motion detector and the physiological parameter detector. The processing module establishes a series of workout stages having varying exercise intensities, estimates at least one of a maximum oxygen uptake quantity (\dot{V}O₂max) and an anaerobic threshold (AT) of the user performing exercise with reference to data obtained by the motion detector and the physiological parameter detector, and generates target workout information to the user performing exercise.

### BACKGROUND OF THE INVENTION

1. Field of the Invention

The present invention relates to a mobile phone apparatus for performing sports physiological measurements and generating target workout information.

2. Description of the Related Art

The use of physiological parameters to gauge physical fitness, and to enhance the effectiveness and safety of exercise has become widespread. As an example, most fitness clubs offer exercise equipment capable of performing such measuring of physiological parameters. The obtained data may then be used to establish target zones in line with desired exercise goals, such as weight reduction and increasing cardiorespiratory fitness. Many portable devices have also been developed that are capable of measuring of physiological parameters. Such portable devices are particularly useful when exercising outdoors. Some portable devices are able to store data and output the same to, for example, a personal computer.

Referring to FIG. 1, a conventional apparatus for performing sports physiological measurements and providing workout support includes a processor (CPU) 201, a clock circuit 202, a keypad 203, an alarm device 204, a display 205, a read only memory (ROM) 206, a random access memory (RAM) 207, a bus 208, a pulse rate detector 209, and a body motion detector 210.

The processor 201 controls the other elements via the bus 208. The ROM 206 stores program instructions executed by the processor 201. The RAM 207 temporarily stores obtained data, as well as data resulting from calculations conducted by the processor 201. The pulse rate detector 209 detects the pulse rate of the user performing exercise. The body motion detector 210 may be configured as an accelerometer, and is used to detect movement by the user performing exercise. A detector interface 211 samples analog output of the pulse rate detector 209 and the motion detector 210, converts the sampled data into digital signals, and provides the digital signals to the processor 201 via the bus 208. The keypad 203 allows for user input of settings for the apparatus, in addition to various personal information, such as height, weight, and sex. The alarm device 204 is controlled by the processor 201 to emit sounds for alerting the user to various situations, such as exceeding a recommended heart rate level. The display 205 displays various information to the user by control of the processor 201. The clock circuit 202 is used to keep track of elapsed time. In comparing the above apparatus with a conventional mobile phone, nearly all the components may be used interchangeably. Therefore, by adding to the conventional mobile phone the necessary detectors and associated program instructions, the mobile phone may be equipped to perform sports physiological measurements and provide workout support.

An example of such a device is disclosed in U.S. Pat. No. 6,817,979 ('979patent), entitled “System and Method for Interacting With a User's Virtual Physiological Model Via a Mobile Terminal.” In the '979 patent, various physiological data are acquired from a user performing exercise in real-time through use of a mobile communication device, and the data are used to generate fitness data. The physiological data are transmitted by the mobile communication device to a network server, which integrates the physiological data into a virtual physiological model of the user.

The '979 patent, however, is not without drawbacks. For example, in the specification of the '979 patent, there is no disclosure with respect to the estimation of maximum oxygen uptake quantity (\dot{V}O₂max) and anaerobic threshold (AT). These two measures are widely used by exercise physiologists as a predictor of performance in sports requiring endurance, and are highly helpful in establishing an effective and safe exercise regimen.

In addition, the system of the '979 patent includes a fitness data engine that is operable at a network server. That is, the fitness data engine, which is supported by the network server, processes all data obtained by the mobile communication device of the '979 patent. This complicates the structure and operation of the network server, and places a greater processing burden on the same.

### SUMMARY OF THE INVENTION

Therefore, the object of this invention is to provide a mobile phone apparatus for performing sports physiological measurements and generating target workout information, in which maximum oxygen uptake quantity (\dot{V}O₂max) and anaerobic threshold (AT) may be easily and effectively measured, and used to provide workout support.

The mobile phone apparatus for performing sports physiological measurements and generating target workout information, according to this invention comprises: a motion detector for detecting motion of a user performing exercise; a physiological parameter detector adapted to be placed in contact with the body of the user performing exercise, the physiological parameter detector detecting at least one physiological parameter of the user performing exercise; a portable housing, the physiological parameter detector being mounted at least partially external to the portable housing; and a processing module mounted in the portable housing and coupled to the motion detector and the physiological parameter detector.

The processing module includes: a workout training program for establishing a series of workout stages having varying exercise intensities to be targeted by the user performing exercise, a performance estimating monitor for estimating at least one of a maximum oxygen uptake quantity (\dot{V}O₂max) and an anaerobic threshold (AT) of the user performing exercise with reference to data obtained by the motion detector and the physiological parameter detector, and a target performance indicator for generating target workout information to the user to performing exercise with reference to data obtained by the motion detector and the physiological parameter detector, as well as the workout training program.

### BRIEF DESCRIPTION OF THE DRAWINGS

Other features and advantages of the present invention will become apparent in the following detailed description of the preferred embodiment with reference to the accompanying drawings, of which:

FIG. 1 is a schematic block diagram of a conventional apparatus for performing sports physiological measurements and providing workout support;

FIG. 2 is a simplified functional block diagram of a mobile phone apparatus for performing sports physiological measurements and generating target workout information according to a preferred embodiment of the present invention;

FIG. 3 is a schematic block diagram, illustrating an exemplary embodiment of the mobile phone apparatus of FIG. 2;

FIG. 4 is another schematic block diagram of the mobile phone apparatus of FIG. 2, illustrating a connection between a mobile phone assembly and a detecting assembly of the mobile phone apparatus when the detecting assembly is mounted external to the mobile phone assembly;

FIG. 5 shows an example of an Astrand-Rhyming nomogram used in the present invention;

FIG. 6 is a chart showing examples of age correction factors applied to the Astrand-Rhyming nomogram of FIG. 5;

FIG. 7 is a graph showing an exemplary relation between heart rate and exercise intensity;

FIG. 8 is a graph showing an exemplary relation between entropy and exercise intensity;

FIG. 9 are graphs showing the relation between power of heart rate variability and exercise intensity;

FIG. 10 is a flow chart of control processes involved in progressively increasing exercise intensity according to a preferred embodiment of the present invention;

FIG. 11 is a flow chart of control processes involved in measuring maximum oxygen uptake quantity (\dot{V}O₂max) according to a preferred embodiment of the present invention;

FIG. 12 is a flow chart of control processes involved in measuring anaerobic threshold (AT) according to a preferred embodiment of the present invention;

FIG. 13 is a flow chart of control processes involved in providing workout support according to a preferred embodiment of the present invention;

FIG. 14 is a schematic perspective view of a holder according to a preferred embodiment of the present invention;

FIG. 15 shows the holder of FIG. 14 in a state securing a mobile phone apparatus;

FIG. 16 is a schematic view, illustrating the mobile phone apparatus of the present invention in different states of use, such as use during running, and real-time monitoring during exercise via a mobile phone network;

FIG. 17 is a schematic view used to describe how the mobile phone apparatus of the present invention may be used to transmit data to and from a personal computer;

FIG. 18 is graph to illustrate an exemplary use of a linear regression method to determine heart rate deflection point (HRDP) utilizing lactate turning points;

FIG. 19 is a graph to illustrate an exemplary use of a third-order curvilinear regression method (Dmax)to determine HRDP; and

FIG. 20 is a graph to illustrate an exemplary logistical growth function for determining HRDP.

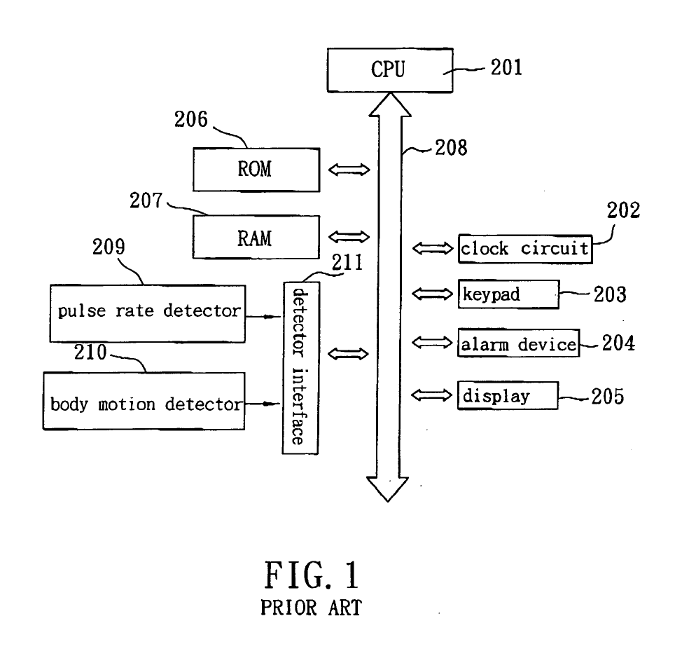

### DETAILED DESCRIPTION OF THE PREFERRED EMBODIMENT

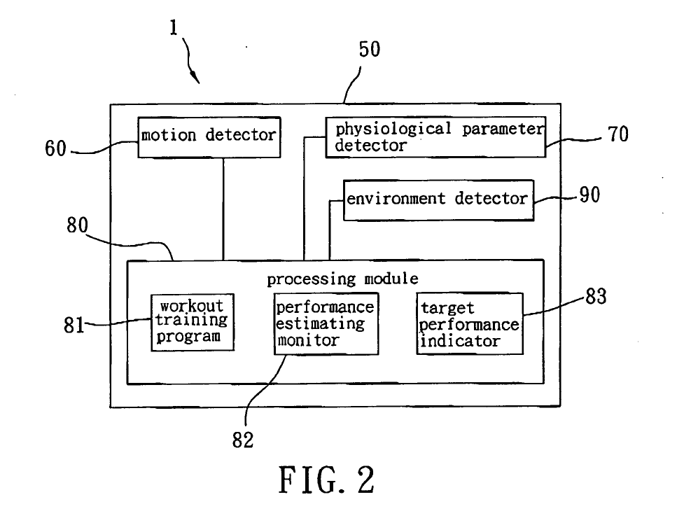

FIG. 2 is a simplified functional block diagram of a mobile phone apparatus 1 for performing sports physiological measurements and generating target workout information according to a preferred embodiment of the present invention.

The mobile phone apparatus 1 includes a portable housing 50, a motion detector 60, a physiological parameter detector 70, a processing module 80, and an environment detector 90. The motion detector 60 detects motion of a user performing exercise, and may be mounted in or externally of the portable housing 50. The physiological parameter detector 70 is mounted at least partially external to the portable housing 50, and is adapted to be placed in contact with the body of the user performing exercise. The physiological parameter detector 70 detects at least one physiological parameter of the user performing exercise. The processing module 80 is mounted in the portable housing 50, and is coupled to the motion detector 60 and the physiological parameter detector 70. The processing module 80 includes a workout training program 81 for establishing a series of workout stages having varying exercise intensities to be targeted by the user performing exercise, a performance estimating monitor 82 for estimating at least one of a maximum oxygen uptake quantity (\dot{V}O₂max) and an anaerobic threshold (AT) of the user performing exercise with reference to data obtained by the motion detector 60 and the physiological parameter detector 70, and a target performance indicator 83 for generating target workout information to the user performing exercise with reference to data obtained by the motion detector 60 and the physiological parameter detector 70, as well as the workout training program 81.

The environment detector 90 is coupled to the processing module 80, and is operable so as to detect environmental conditions to obtain environmental data. The performance estimating monitor 82 suitably factors in the environmental data when estimating the \dot{V}O₂max and the AT of the user performing exercise.

In this embodiment, the processing module 80 includes a processor and a program memory coupled to the processor. The program memory stores program instructions executed by the processor for configuring the processing module 80 to include the workout training program 81, the performance estimating monitor 82, and the target performance indicator 83.

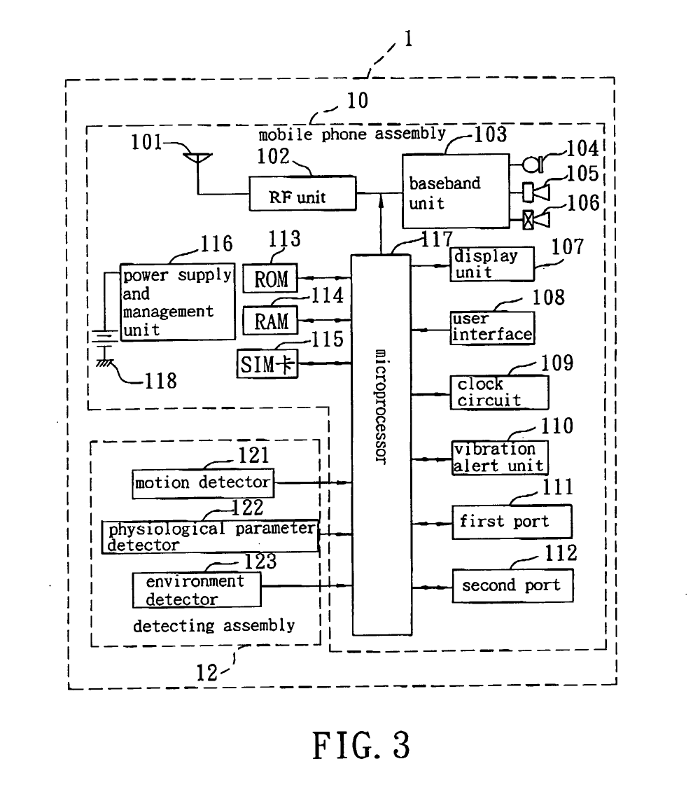

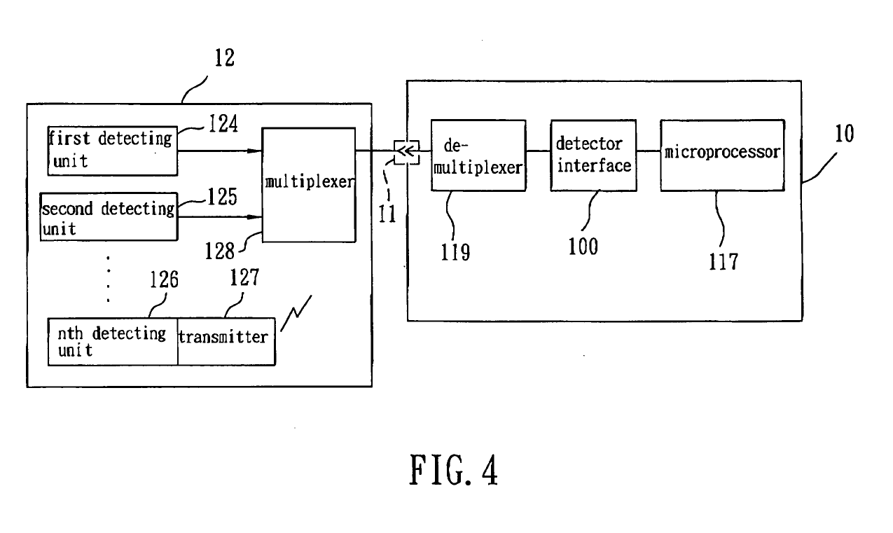

FIGS. 3 and 4 show an exemplary embodiment of the mobile phone apparatus 1 of FIG. 2. In this example, the mobile phone apparatus 1 includes a mobile phone assembly 10, a transmission line 11 (see FIG. 4), and a detecting assembly 12 having a motion detector 121, a physiological parameter detector 122, and an environment detector 123. The mobile phone assembly 10 includes a microprocessor 117 such as a digital signal processor, a read only memory (ROM) 113, a random access memory (RAM) 114, a subscriber identity module (SIM) card 115, a power supply and management unit 116 having a battery 118, an antenna 101, a radio frequency (RF) unit 102 having a transmitter, a receiver, and a frequency synthesizer (all not shown), a baseband unit 103, a microphone 104, a buzzer unit 105, a speaker 106, a display unit 107 configured as a liquid crystal display, a user interface 108 configured as a keypad, a clock circuit 109, a vibration alert unit 110, a first port 111 such as an RS-232 port or a USB port for wired communications, a second port 112 such as an infrared port or Bluetooth port for wireless communications, a detector interface 100 (see FIG. 4), and a de-multiplexer 119 (see FIG. 4).

The ROM 113 includes program instructions that are executed by the microprocessor 117 to enable full duplex telecommunications by the mobile phone assembly 10 in a conventional manner, as well as to perform sports physiological measurements and to generate target workout information. The microprocessor 117 performs overall control of the mobile phone apparatus 1, in addition to executing the program instructions stored in the ROM 113. The RAM 114 temporarily stores data input by the user performing exercise, as well as results of calculations performed by the microprocessor 117. The microprocessor 117 controls the microphone 104, the buzzer unit 105, and the speaker 106 through the baseband unit 103. The first port 111 and the second port 112 may be used transmit and receive data to and from a personal computer (to be described hereinafter).

The motion detector 121 may be configured as an accelerometer, and functions to detect movement of the user performing exercise. The motion detector 121 is able to convert movement of the user performing exercise to electrical signals in a conventional manner. The converted electrical signals may then be used to calculate number of paces per unit time, motion speed, distance traveled, and work exerted by the user performing exercise in power units. This will be described in greater detail below.

The physiological parameter detector 122 is used to detect physiological parameters of the user performing exercise. The physiological parameter detector 122 detects heart rate, pulse rate, blood pressure, body temperature, respiratory rate, etc. Different detectors may be used for the different measurements. For example, when \dot{V}O₂max and AT are measured by an indirect method (to be described below), a pulse rate detector (not shown) may be used. To simplify detection, the obtained pulse rate may be considered equivalent to the heart rate (beats/min) of the user performing exercise. The pulse rate may be measured in a variety of ways, such as by using a piezoelectric detector to take a radial pulse, or by using an optical pulse reader that measures movement of blood in the capillaries of the finger. Since these and other techniques are well known in the art, a detailed description thereof will be omitted herein for the sake of brevity.

The environment detector 123 is operable so as to detect environmental conditions to obtain environmental data representative of, for example, ambient temperature and air pressure. The microprocessor 117 factors in the environmental data when estimating the \dot{V}O₂max and the AT of the user performing exercise in a manner to be described hereinafter.

The physiological parameter detector 122 is mounted at least partially external to the mobile phone assembly 10 to allow for contact with the body of the user performing exercise. The environment detector 123 and the motion detector 121 may be mounted in or external to the mobile phone assembly 10. Further, the motion detector 121, the physiological parameter detector 122, and the environment detector 123 are coupled to the microprocessor 117 through the detector interface 100 (see FIG. 4). When these elements are externally mounted, the detector interface 100 may allow for a wired or wireless connection for coupling to the microprocessor 117.

FIG. 4 shows the connection between externally disposed detecting units and the mobile phone assembly 10. A plurality of detecting units, e.g., a first detecting unit 124, a second detecting unit 125, and an nth detecting unit 126, maybe coupled through a wired or wireless connection to the microprocessor 117 of the mobile phone assembly 10.

In the example shown in FIG. 4, the first and second detecting units 124,125 are coupled via a wired connection to the microprocessor 117 through a multiplexer 128, the transmission line 11, the de-multiplexer 119, and the detector interface 100, the latter two of which are part of the mobile phone assembly 10. The nth detecting unit 126, on the other hand, is wirelessly coupled to the microprocessor 117 of the mobile phone assembly 10 through the detector interface 100. In this case, a transmitter 127 is associated with the nth detecting unit 126. The transmitter 127 wirelessly transmits signals to the detector interface 100 in any known manner, such as through inductive coupling, infrared transmission, microwave transmission, radio frequency transmission, Bluetooth transmission, etc.

The theory and principles behind the operation and use of the mobile phone apparatus 1 will now be described. At the onset, user-specific data, such as height, weight, age and sex of the user performing exercise, are inputted through the keypad 108 or through conventional data transmission techniques for storage in the RAM 114 upon switching the mobile phone apparatus 1 to a mode for performing sports physiological measurements and for generating target workout information. Exercise measurements are performed using the motion detector 121, that is, by measuring the physical movement of the user performing exercise. The data obtained by the motion detector 121 may be used by the microprocessor 117 to determine speed, displacement, work, and power. Power is equivalent to exercise intensity, and its units are kp-m/min (i.e., kilopond-meters per minute). The kp-m/min unit corresponds to the kg-m/min unit (kilogram-meter per minute).

The motion detector 121 is secured to a specific area of the body of the user performing exercise. The motion detector 121 generates analog voltage pulse signals corresponding to the level of acceleration. When the motion detector 121 is positioned on the wrist of the user, for example, the motion detector 121 outputs voltage pulse signals corresponding to acceleration resulting from the swinging of the user's arms during exercise. Through frequency analysis and using a fast Fourier transform (FFT) algorithm, the required signals may be obtained from the pulse signals, then converted to calculate, for example, the number of paces per unit time. Stride of the user performing exercise is indicated in units of meters per step (m/step). By multiplying stride by the number of steps per unit time, speed may be obtained in units of meters per minute (m/min). By multiplying the speed by the weight of the user performing exercise, exercise intensity may be obtained in units of kilopond-meters per minute (kp-m/min) or kilogram-meters per minute (kg-m/min) as described above.

Stride (m/step) may be obtained directly through user input and stored in the RAM 114. Alternatively, stride may be obtained indirectly as a function of the height of the user performing exercise, or as a function of both the height and weight of the user performing exercise. Using the knowledge that stride varies with speed, adjustments to the input or calculated stride may be made according to the calculated speed of the user performing exercise. The mobile phone apparatus 1 of the present invention is able to calculate \dot{V}O₂max and AT of the user performing exercise. These two indices may then be used to provide workout support if desired. \dot{V}O₂max and AT are extremely important in sports physiology and allow for the quantitative evaluation of an individual's endurance. That is, \dot{V}O₂max and AT may be used to quantitatively measure fitness, as well as to quantify, compare, and confirm the effects of training.

In exercise physiology, maximum oxygen uptake quantity (\dot{V}O₂max) indicates the maximum rate of oxygen that can be utilized by the body during severe exercise. The measurement is ideally taken at sea level. \dot{V}O₂max provides an indication of cardio-respiratory endurance. By dividing \dot{V}O₂max by the weight of the user, a relative value (ml/kg/min) may be obtained, which is an internationally recognized standard measure of an individual's cardio-respiratory fitness.

In addition to quantitatively evaluating an individual's endurance and confirming the effects of exercise training, \dot{V}O₂max may also function as an index of exercise training load. For example, by exercising at an exercise intensity of 50-85% of an individual's \dot{V}O₂max, it is believed (by exercise physiologists) that the greatest benefit from exercise may be obtained. Anaerobic threshold (AT) is an important indicator in exercise physiology and is defined as the point at which lactate (lactic acid) begins to accumulate in the bloodstream. AT is obtained by taking measurements in a known manner while the intensity of exercise is incrementally increased. AT defines the boundary between aerobic and anaerobic systems of the human body.

AT may also be used to quantitatively evaluate an individual's endurance, confirm the effects of training, and as an index of exercise training load similar to the manner in which \dot{V}O₂max is used. According to recent research, AT is believed to be a better measure of endurance than \dot{V}O₂max.

The method of measuring \dot{V}O₂max will now be described. \dot{V}O₂max may be measured directly or indirectly. In the direct method, which is typically performed in a laboratory setting, exhalation amounts are measured while the test subject is undergoing an incrementally intensive exercise load. By taking the ratio of the oxygen content to carbon dioxide content in the air exhaled by the subject, the maximum oxygen uptake quantity per minute may be obtained. A drawback of such a direct method, however, is that it is necessary for the test subject to eventually reach a level of maximum exercise intensity. This is not suitable for all persons (e.g., children and older people).

Therefore, an indirect method has been devised by specialists in the field to replace the direct method of measuring \dot{V}O₂max. In the indirect method, the test subject need not exercise to a maximum intensity, thereby allowing for safe application to all persons. The indirect method is particularly useful when no medical professionals are present for the test, the physical condition of the test subject is unknown, and/or the test subject leads a sedentary lifestyle and is not used to exercising to maximum intensity.

In the indirect method, the subject undergoes a progressively increasing exercise intensity to a sub-maximal level, during which physiological parameters are measured, e.g., heart rate. Next, \dot{V}O₂max is estimated using a look-up table, an Astrand-Rhyming nomogram, or a mathematical formula.

Since the heart rate (beats/min) of a normal person is equivalent to his or her pulse rate, the pulse rate is commonly used as a measure of heart rate due to the relative ease of measuring the former, particularly during exercise.

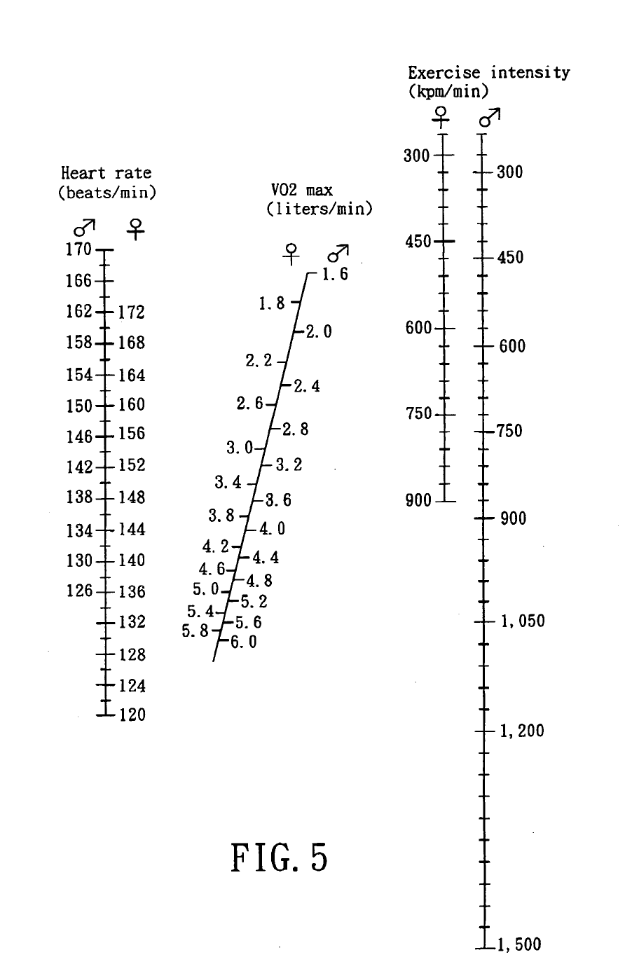

FIG. 5 shows an example of an Astrand-Rhyming nomogram that is used to estimate \dot{V}O₂max. The heart rate is plotted on the left axis, and the exercise intensity is plotted on the pair of right axes. \dot{V}O₂max is plotted on a straight, inclined axis between the heart rate and exercise intensity axes. Gender symbols are shown at the top of each of the axes (pair of axes for exercise intensity) to indicate the different measurements used for different sexes. The Astrand-Rhyming nomogram is formed on the presumption that there is a linear relation between a test subject's exercise intensity and heart rate. For this reason, when using the Astrand-Rhyming nomogram to measure \dot{V}O₂max, it is necessary to first verify that there is such a linearly increasing relation between the exercise intensity and the heart rate of the particular test subject.

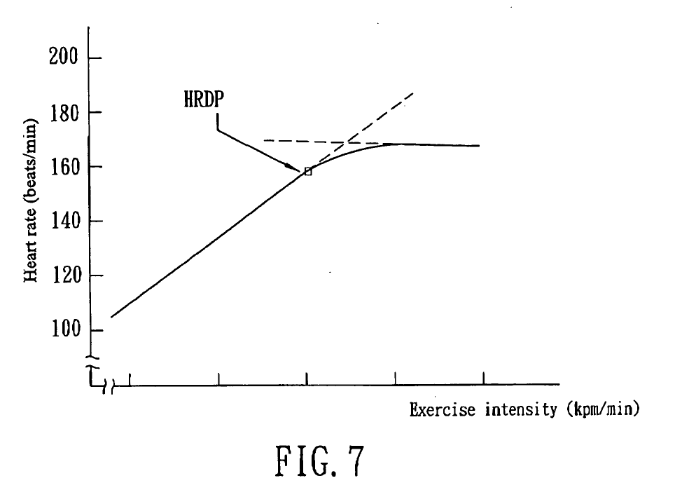

Referring to FIG. 7, an example of the relation between the heart rate and exercise intensity of a test subject is shown in a graph. In general, when exercise intensity is below a specific level, the relation between the heart rate and exercise intensity is such that heart rate increases in direct proportion to exercise intensity as shown in FIG. 7. When the exercise intensity of the test subject exceeds a specific level, however, such a linear relation ceases to exit, with the rate of increase in the heart rate becoming less than the rate of increase in exercise intensity. Eventually, saturation occurs and additional increases in heart rate are no longer possible.

The point at which the linear relation between the heart rate and exercise intensity ends is commonly referred to as the heart rate deflection point (HRDP). The straight line upto the HRDP is related to the fitness level of the test subject. That is to say, the slope of the straight line provides an indication of the overall fitness of the test subject. For example, measurements for a first test subject that result in a more horizontal line than measurements for a second test subject indicates that the first test subject is able to undergo a greater exercise intensity at the same heart rate level than the second test subject.

The indirect method for estimating \dot{V}O₂max involves progressive incremental exercise testing during which heart rate is measured. In order to determine whether a linear relationship exists, it is necessary to measure exercise intensities at a minimum of three points, and determine the heart rate at each point. The obtained exercise intensity-heart rate pairs have to confirm the existence of a straight-line relationship by linear regression analysis.

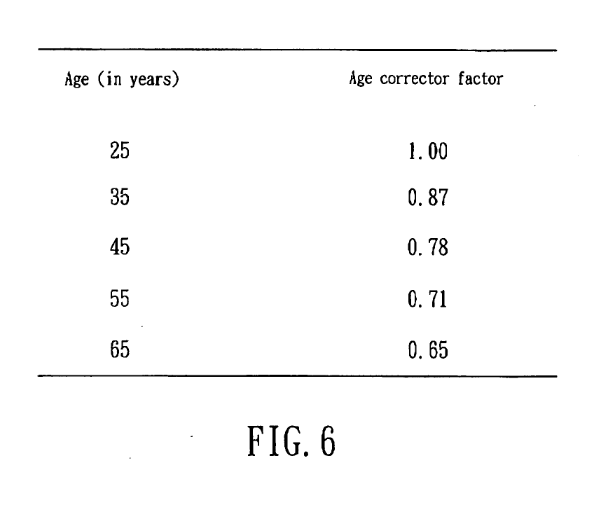

After determining that a linear relation between exercise intensity and heart rate exists, it is necessary to know only the gender of the test subject in order to determine \dot{V}O₂max by applying the obtained data to an Astrand-Rhyming nomogram, such as that shown in FIG. 5. In addition, since \dot{V}O₂max varies according to age, \dot{V}O₂max maybe adjusted by an age correction factor. FIG. 6 shows a chart of various age correction factors that may be applied to the Astrand-Rhyming nomogram of FIG. 5.

The method of measuring anaerobic threshold (AT) will now be described. AT is defined as the point at which lactic acid begins to accumulate in the bloodstream as explained above. The determination of AT involves at least one of measuring lactate in the blood, gas exchange, and heart rate: AT obtained by measuring lactate in the blood is referred to as lactate threshold, AT obtained by measuring gas exchange is referred to as ventilatory threshold, and AT obtained by measuring heart rate is referred to as heart rate threshold. AT determined by measuring heart rate is the simplest. Regardless of which method is used, measurements are performed while the test subject is undergoing exercise of a progressive intensity. In the gas exchange method, carbon dioxide in the air exhaled by the test subject, as well as the oxygen in the air inhaled by the test subject, are measured. The information is then provided to a computer to determine AT. This final method is referred to as a V-slope method.

Referring again to FIG. 7, heart-rate threshold may be determined from such a plot of heart rate versus exercise intensity. Conconi et al. developed the concept of heart-rate threshold. They found that when exercise intensity is below a specific level, there is a linear relation between heart rate and exercise intensity. However, when exercise intensity exceeds a given value (i.e., the specific level), increases in the heart rate slow down until saturation occurs. That is, after this specific value referred to as HRDP as mentioned above, the linear relation between heart rate and exercise intensity ceases to exist. Although slightly higher than the true anaerobic threshold value, the HRDP is viewed to be roughly equivalent thereto.

One common exercise test used to estimate AT is the Conconi test, which is a simple, convenient and non-invasive test. In this method, exercise intensity is progressively increased, during which heart rate is monitored and recorded. The resulting HRDP is determined to be the heart rate threshold.

A variety of methods of determining HRDP have been developed in order to improve the precision of estimating AT therefrom. The methods have developed from simple visual inspection to more recent methods involving computer-aided regression analysis. Regression techniques include simple linear regression, third-order curvilinear regression, and logistical growth function. Regardless of which method is used, HRDP is estimated.

In each of the following examples for determining HRDP, exercise load is progressively increased at increments of 15 W (1 W=6.12 kp-m/min) and at two-minute intervals after a test subject has completed a warm-up exercise stage (e.g., 50 W exercise intensity for S minutes) until maximal exercise intensity and heart rate levels are reached.

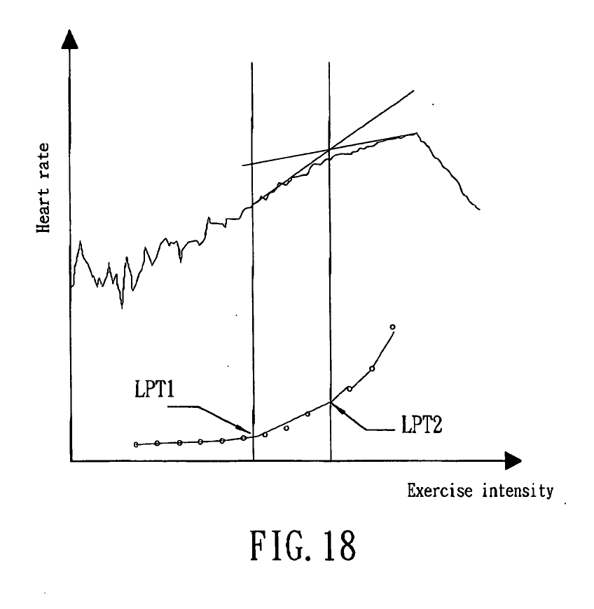

FIG. 18 shows a graph used to illustrate the linear regression method of calculating HRDP. In the graph, a first lactate turn point (LTP1) and a second lactate turn point (LTP2) are utilized, in which the LTP2 is the AT value. A second-degree polynomial represents a heart rate curve in this method. Two minimum standard deviation regression lines are drawn through data points of the LTP1 and of maximum exercise intensity, and the heart rate corresponding to the point at which these two regression lines intersect is the HRDP. It may be determined if the heart rate curve is increasing or decreasing by examination of the slopes of these two regression lines.

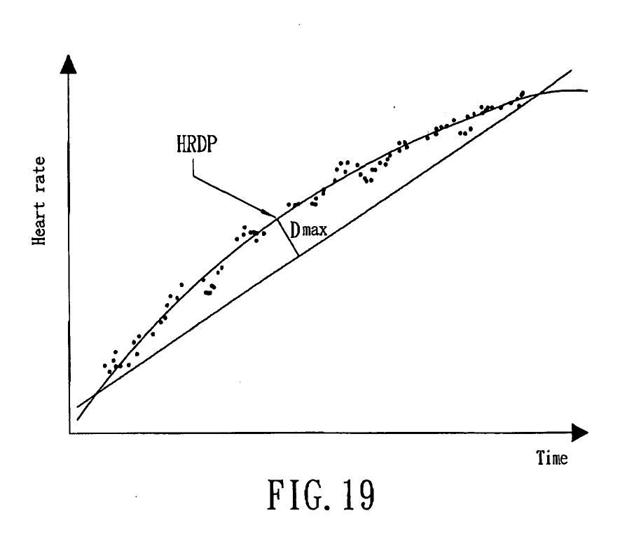

FIG. 19 shows a graph used to illustrate the third-order curvilinear regression method (Dmax method) of calculating HRDP. Heart rate data as a function of time is first plotted on the graph. The points on the graph are then used to obtain the heart rate regression curve. Next, ends of the regression curve are connected by a straight line. The heart rate corresponding to the point on the regression curve farthest from the straight line is the HRDP.

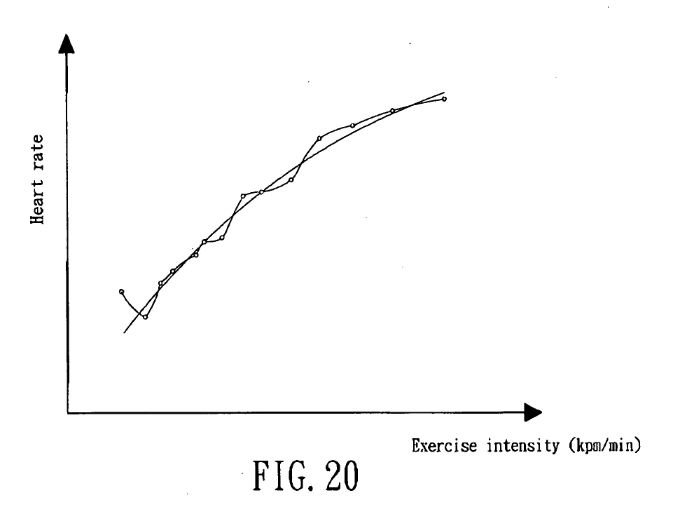

FIG. 20 shows a graph used to illustrate the logistical growth function method of calculating HRDP. The logistical growth function is commonly used in establishing a growth rate model for biological organisms since slowing growth with eventual saturation is typical of the growth experienced with such biological organisms. The logistical growth function [y=1/(abx+c)] into which are input heart rate and exercise intensity is a basic computer program. The logistical growth function can generate a heart rate curve that increases at a specific growth rate until reaching the HRDP, where the rate of increase in the curve decreases until reaching the maximum heart rate.

If heart rate is (H) and exercise intensity is (P) the logistical growth function may be expressed by the equation Hₚ=1/(ab^{P}+(1/m)), where m is the maximum heart rate, a is the y-intercept, and b is the slope. This equation may be re-arranged to obtain the linear equation, (1/H_{P})−(1/m)=ab^{P}. By multiplying both sides of this linear equation by the natural log, the following equation may be obtained: ln((1/H_{P})−(1/m))=lna+(lnb)P. Therefore, the linear equation of the logistical growth function may be used in regression analysis to convert data (i.e., heart rate and exercise intensity data).

As shown in FIG. 20, the upper portion of the obtained logistical growth function curve is converted into a derivative curve for use in performing calculation and analysis. The y-axis coordinates and their corresponding x-axis coordinates (exercise intensity) of the derivative curve may be used to obtain the slope of the given curve (i.e., the logistical growth function curve). Therefore, exercise intensity and heart rate may be obtained from a specific y-axis coordinate of the derivative curve, and this particular point represents the HRDP. In order to prevent drift in the analyzed curves, it is necessary that the starting points of exercise intensity values remain constant.

The concept of analyzing heart rate variability during exercise to measure the AT point has been developed in recent times. Heart rate variability involves analysis of changes in R-R interval times associated with consecutive heart beats, in which the R-R interval time is the heart beat cycle whereas the heart rate is its inverse.

The theory and method of utilizing entropy (E) to determine AT will now be described. After performing a warm-up exercise (e.g., 5 minutes of exercise at an intensity of 50 W, where 1 W=6.12 kp-m/min), the test subject progressively increases exercise intensity, for example, at a rate of 15 W every two minutes. At the same time, the R-R interval value (i.e., the period of one heart beat in units of milliseconds) of the heart rate signals is measured, and indicated as R-R(n), where n is the continuous number of heart beats. Further, the inverse of the period of one heart beat cycle is the heart rate (beats/min).

A continuous heart beat cycle value is defined as [R-R(n)−R-R(n+1)], where 1 n N−1, N indicating the total number of heart beats within a predetermined time period. An increase in the heart rate indicates that the current heart beat cycle has shortened relative to the previous cycle. Therefore, the continuous heart beat cycle value is positive when the heart rate increases, and is negative when the heart rate decreases.

In addition, the above continuous heart beat cycle may be indicated by a percent index (PI). PI may be obtained by the following Equation 1:
PI(n)%=[R-R(n)−R-R(n+1))/R-R(n)×100% (1)
where 1 n N−1.

In order to obtain more precise data, the last 100 heart rates may be used to calculate PI.

Frequency f(i) indicates the number of times PI(n) occurs within a predetermined interval, where “i” is an integer. Furthermore, probability p(i) may be obtained by Equation 2 below:

$$\begin{matrix}p ( i ) = f ( i ) / f \\ where f = {\sum }_{i} f ( i ) .\end{matrix} \qquad ( 2 )$$

Therefore, entropy may be defined by the following

$$Equation 3 : \\ E = {- \sum }_{i} p ( i ) {log}_{2} p ( i ) \qquad ( 3 )$$

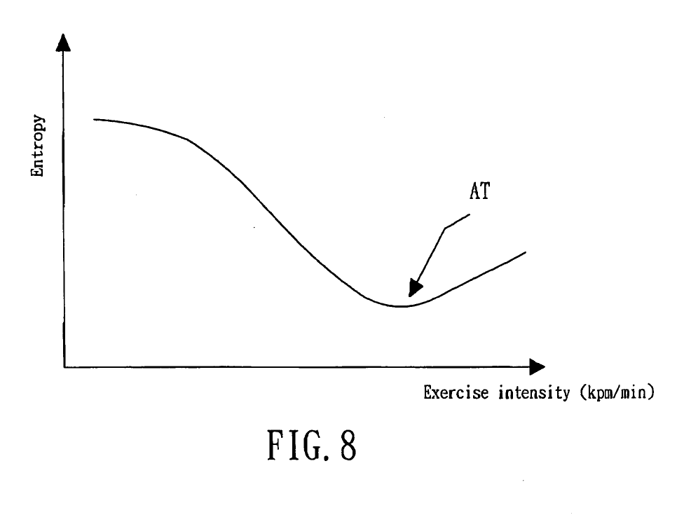

FIG. 8 is a graph showing the relation between entropy and exercise intensity. In the graph, an increasing exercise intensity accompanied by a decreasing entropy indicates that the test subject has not reached AT. In order to obtain AT, exercise intensity must be progressively increased, during which the PI values and entropy are measured. The point at which entropy begins to increase indicates the AT of the test subject.

The theory and method for determining AT using the power of heart rate variability will now be described.

After performing a warm-up exercise (e.g., 5 minutes of exercise at an intensity of 50 W, where 1 W=6.12 kp-m/min), the test subject progressively increases exercise intensity, for example, at a rate of 15 W every two minutes. At the same time, the R-R interval value (i.e., the period of one heart beat in units of milliseconds) of the heart beat signals is measured, and indicated as R-R(n), where n is the continuous number of heart beats. Further, the inverse of the period of one heart beat cycle is the heart rate (beats/min).

The power of heart rate variability (i.e., Power(n) with units of ms²) is the square of the difference between consecutive heart beat cycle values. Power(n) may be obtained by the following Equation 4:
Power(n)=(R-R(n)−R-R(n+1)]² (4)
where 1 n N−1, N indicating the total number of heart beats within a predetermined time period.

Next, the average value of Power(n) within a unit time is calculated. The unit time may be, for example, 30-second periods within a 2-minute interval. Therefore, the exercise load may be progressively increased at intervals until the maximum heart rate is reached.

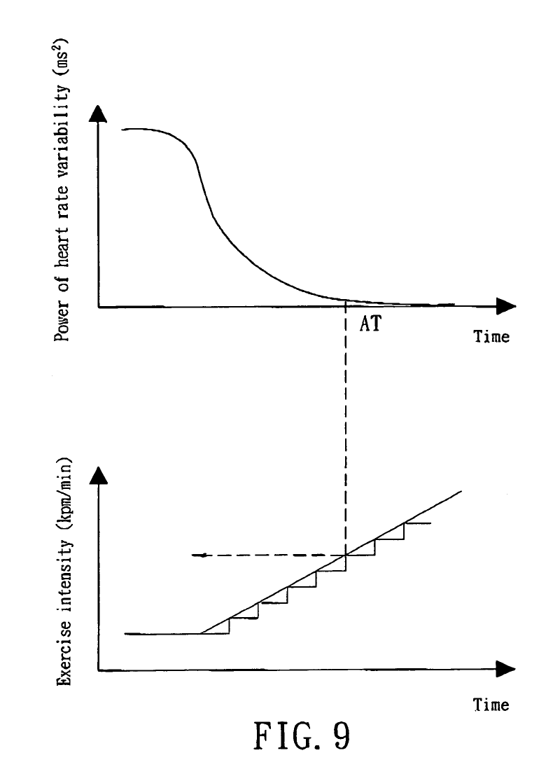

Referring to FIG. 9, a curve can be obtained through regression analysis of the average values of Power(n) thus obtained. As evident from FIG. 9, the power of heart rate variability [Power(n)] decreases with increases in exercise intensity until it is approximately zero. This characteristic can be used to estimate the an aerobic threshold (AT) point. In particular, the AT is point is one where the Power(n) value is lower than a preset lower limit, and the slope [Power(n−1)−Power(n)] is lower than a preset value.

Regardless of which method is used to estimate \dot{V}O₂max or AT, measurements of physiological parameters are performed while the test subject is undergoing exercise of a progressive intensity. Testing is typically performed using machines that allow for precise settings, such as a bicycle ergometer, a treadmill, or a step machine.

In the past, measurements were performed using the Conconi method in which a “fixed distance, fixed amount” mode was used while increasing speed. However, the “fixed distance” is now commonly replaced with a “fixed interval (of time).” In the present invention, an accelerometer fixed at a suitable position of the user's body and an indication mechanism (i.e., the target performance indicator 83 of FIG. 2) are used to simulate the use of exercise equipment in a laboratory setting. The user performing exercise is alerted to progressively increase his or her exercise intensity at fixed intervals and by a fixed amount. In other words, after converting the electrical pulse signals obtained from the accelerometer attached to, for example, the user's wrist, the number of paces per unit time (i.e., steps/min) may be obtained. Furthermore, by multiplying the number of paces per unit time (steps/min) by stride (m/step), speed (m/min) may be obtained. Next, by multiplying the weight of the user by the speed, exercise intensity may be obtained in units of kilogram-meters per minute (kg-m/min) or kilopond-meters per minute (kp-m/min). Therefore, through the indication mechanism of the present invention, the user performing exercise may be instructed to adjust the number of paces per unit time to thereby effect changes in exercise intensity. At the same time, by establishing workout stages, exercise intensity may be progressively increased at fixed intervals and by a fixed amount.

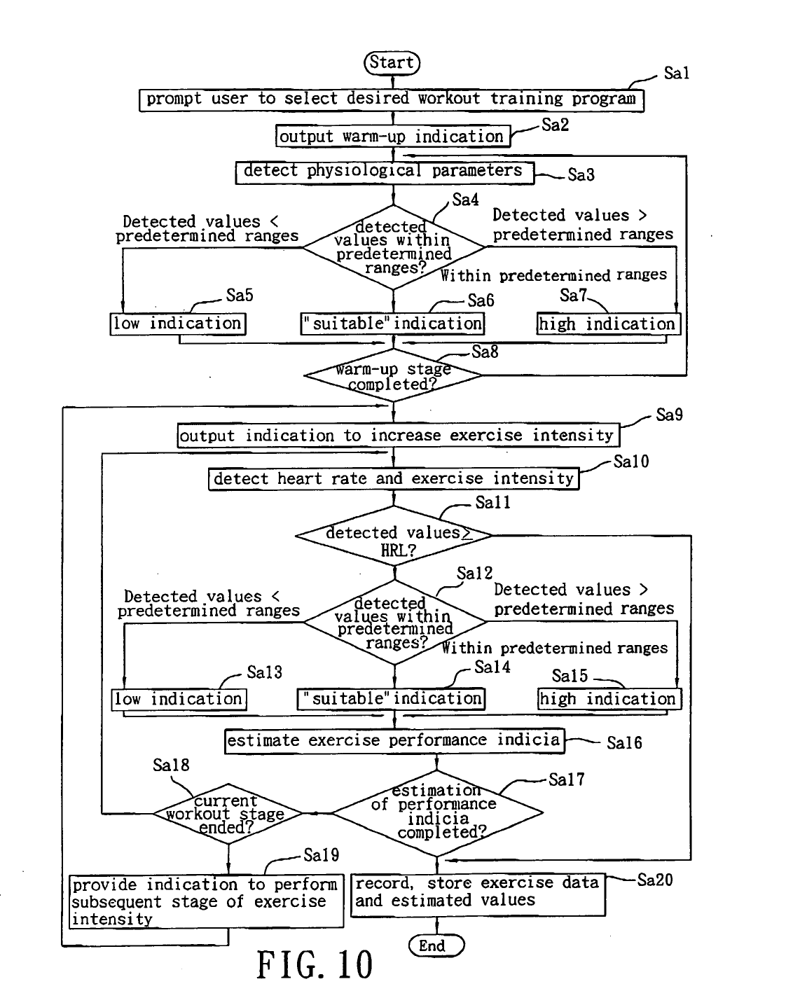

Referring to FIG. 10, the use and operation of the mobile phone apparatus 1 of the present invention will now be described. For the following discussion, it is assumed that the mobile phone apparatus 1 is configured as the mobile phone shown in FIG. 3.

First, in step Sa1, the user is prompted by control of the microprocessor 117 to select a desired workout training program through manipulation of the user interface 108. As an example, there maybe provided five levels of workout training programs, in which the higher the level, the greater will be the increase in exercise intensity for the different workout stages. If it is further assumed for this example that each workout stage lasts two minutes, that a first level has been selected, and exercise intensity increases at a rate of 20 W (where 1 W=6.12 kp-m/min) for the first level, then the user performing exercise must increase exercise intensity by 20 W every two minutes following a period of warm-up exercise as described below.

Next, in step Sa2, the microprocessor 117 of the mobile phone apparatus 1 performs control to output a warm-up indication to the user performing exercise. The progressive exercise intensity process of this invention includes a warm-up stage in which the user performs exercise at a predetermined exercise intensity for a predetermined time. As an example, the warm-up stage may involve exercising for five minutes at an exercise intensity of Sow.

Subsequently, in step Sa3, the detecting assembly 12 of the mobile phone apparatus 1 detects the user's heart rate and exercise intensity during the warm-up stage. Next, in step Sa4, the microprocessor 117 of the mobile phone apparatus 1 compares the detected values with predetermined values, and determines if the detected values are within predetermined ranges, exceed the predetermined ranges, or are lower than the predetermined ranges. If the detected values exceed the predetermined ranges, then an indication is provided to the user in step Sa7 that the actual exercise intensity is too high. If the detected values are lower than the predetermined ranges, then an indication is provided to the user in step Sa5 that the actual exercise intensity is too low. Finally, if the detected values fall within the predetermined ranges, then a “suitable” indication is provided to the user in step Sa6. The user performing exercise is able to adjust his or her exercise intensity as needed according to the indications thus provided.

After any of the steps Sa5, Sa6, and Sa7, it is determined if the time interval associated with the warm-up stage has elapsed in step Sa8. If the time interval of the warm-up stage has not elapsed, then the flow returns to step Sa3. However, if the time interval of the warm-up stage has elapsed, the microprocessor 117 performs control in step Sa9 to output an indication to the user performing exercise to begin progressive increases in exercise intensity. Based on this indication, the user starts to increase exercise intensity.

Next, in step Sa10, the detecting assembly 12 detects the heart rate and exercise intensity of the user performing exercise. Subsequently, in step Sa11, the mobile phone apparatus 1 determines if the detected heart rate exceeds the heart rate limit (HRL) of the user, where the HRL is calculated as follows:
HRL=0.85(220−age).

If the detected heart rate exceeds or is equal to the HRL of the user, then step Sa20 is performed and all intervening steps are skipped. In step Sa20, the microprocessor 117 performs control to record and store obtained exercise data and estimated values in the RAM 114, after which an indication may be provided to the user performing exercise to discontinue exercise.

However, if the detected heart rate is less than the HRL in step Sa11, then the microprocessor 117 performs a comparison of the detected heart rate and exercise intensity with corresponding predetermined ranges in step Sa12. If the detected values are less than the predetermined ranges, then an indication is provided to the user performing exercise in step Sa13 that the actual exercise intensity is too low. If the detected values exceed the predetermined ranges, then an indication is provided to the user performing exercise in step Sa15 that the actual exercise intensity is too high. Finally, if the detected values fall within the predetermined ranges, then a “suitable” indication is provided to the user in step Sa14. The user performing exercise may adjust his or her exercise intensity as needed based on the indications thus provided.

After any of the steps Sa13, Sa14, and Sa15, the microprocessor 117 performs calculations based on the obtained exercise data to estimate exercise performance indicia (e.g., \dot{V}O₂max and AT) in step Sa16. During such progressive increases in exercise intensity, following an increase in exercise intensity by a fixed amount for each workout stage, a predetermined exercise intensity is maintained for the duration of the workout stage. This allows for the physiological parameters detected in each workout stage to be more precisely obtained.

Next, in step Sa17, the microprocessor 117 determines if estimation of the exercise performance indicia has been successfully performed. If not, it is determined in step Sa18 if the current workout stage has ended. If the current workout stage has not ended, then the flow returns to step Sa10. However, if the current workout stage has ended in step Sa18, then the microprocessor 117 performs control in step Sa19 to provide an indication to the user performing exercise to perform a subsequent stage of exercise intensity, after which the flow returns to step Sa9. However, if estimation of the exercise performance indicia has been successfully performed in step Sa17, then, in step Sa20, the microprocessor 117 of the mobile phone apparatus 1 records and stores exercise data and estimated values in the RAM 114. Following step Sa20, an indication may be provided to the user performing exercise to discontinue exercise (not shown).

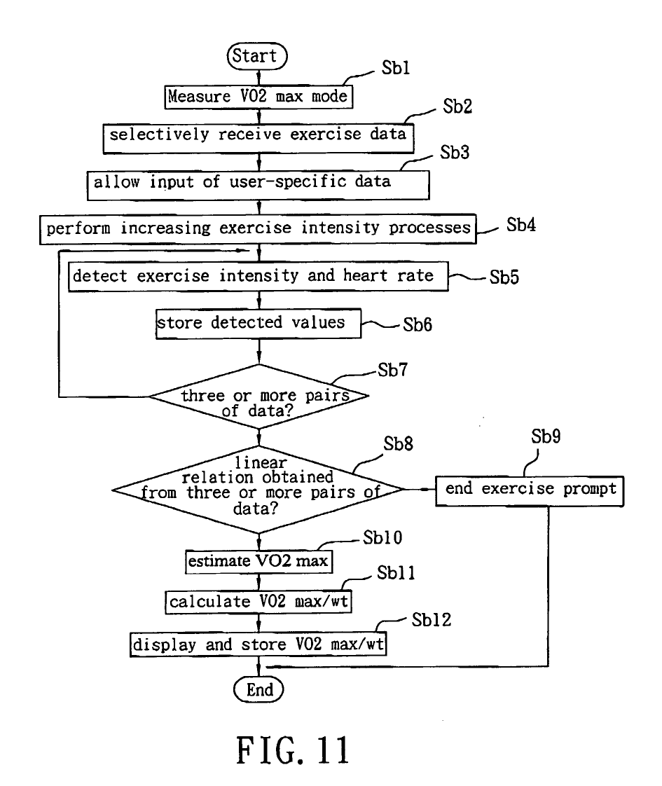

FIG. 11 shows an example of processes involved in estimating \dot{V}O₂max.

First, in step Sb1, the user manipulates the user interface 108 to place the mobile phone apparatus 1 in a measure \dot{V}O₂max mode. Next, in step Sb2, the microprocessor 117 performs control to selectively receive exercise data. In step Sb3, the microprocessor 117 of the mobile phone apparatus 1 performs control to allow for input of user-specific data, such as height, weight, sex, and age. Subsequently, in step Sb4, control processes associated with progressively increasing exercise intensity are performed. Next, in step Sb5, the detecting assembly 12 detects exercise intensity and heart rate. In step Sb6, the microprocessor 117 stores the obtained average exercise intensity data for each workout stage with their corresponding average heart rate data in the RAM 114.

Next, in step Sb7, the microprocessor 117 determines if three or more data pairs of exercise intensity and heart rate have been obtained. This step is performed since there must be at least three data pairs of exercise intensity and heart rate to determine if there is a linearly increasing relationship between these parameters. If there are less than three data pairs of exercise intensity and heart rate, then the flow returns to step Sb5.

However, if three or more data pairs of exercise intensity and heart rate have been obtained, it is determined in step Sb8 if there is a linear relation between the detected exercise intensity and heart rate. If there is no such linear relation, then the mobile phone apparatus 1 outputs an end exercise prompt to the user performing exercise in step Sb9, after which the process is ended.

However, if there is a linear relation between the detected exercise intensity and heart rate in step Sb5, the microprocessor 117 estimates \dot{V}O₂max of the user performing exercise in step Sb10. Adjustments may be made for the estimation of the \dot{V}O₂max, such as by applying an age correction factor. In this embodiment, an Astrand-Rhyming nomogram is used to estimate \dot{V}O₂max in step Sb10, in which the gender of the user performing exercise may be used to obtain a more precise estimation. Subsequently, in step Sb11, the microprocessor 117 calculates \dot{V}O₂max/wt. That is, the estimated \dot{V}O₂max is divided by the user's weight to thereby obtain \dot{V}O₂max/wt (ml/kg/min), which is an internationally recognized standard measure of an individual's cardio-respiratory fitness. Finally, in step Sb12, the microprocessor 117 performs control to display the obtained \dot{V}O₂max/wt value on the display unit 107, and to store the same in the RAM 114.

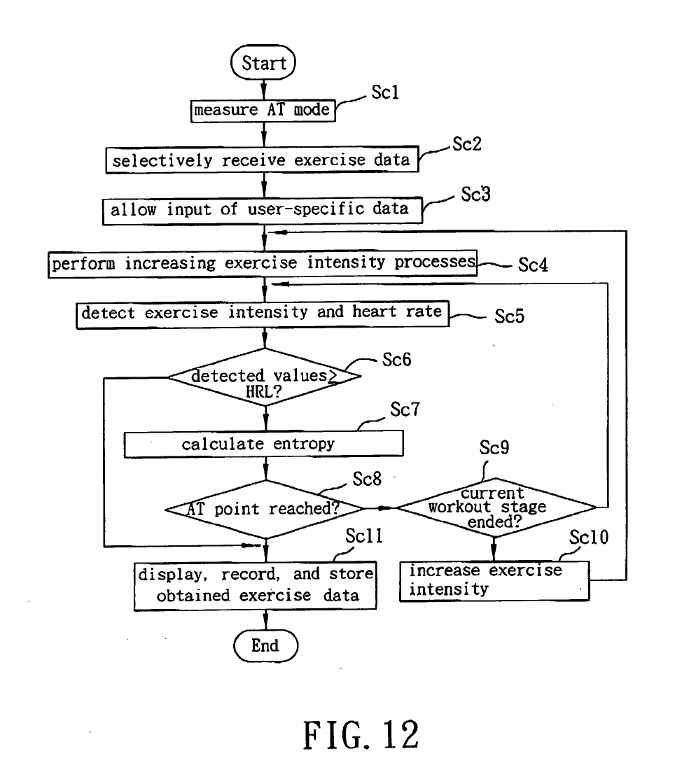

FIG. 12 shows an example of processes involved in estimating anaerobic threshold (AT).

First, in step Sc1, the user manipulates the user interface 108 to place the mobile phone apparatus 1 in a measure AT mode. Next, in step Sc2, the microprocessor 117 performs control to selectively receive exercise data. In step Sc3, the microprocessor 117 performs control to allow for the input of user-specific data, such as height, weight, sex, and age. Subsequently, in step Sc4, control processes associated with progressively increasing exercise intensity are performed. Next, in step Sc5, the detecting assembly 12 detects exercise intensity and heart rate. In step Sc6, the microprocessor 117 of the mobile phone apparatus 1 determines if the heart rate is greater than or equal to the heart rate limit (HRL) of the user. If the heart rate of the user performing exercise is at or exceeds his or her HRL, then step Sc11 is performed, in which the microprocessor 117 performs control to display, record, and store obtained exercise data of the user, after which the process is ended.

However, if the heart rate of the user performing exercise is less than his or her HRL, then entropy is calculated in step Sc7. Next, in step Sc8, the microprocessor 117 determines if the AT point has been reached. If exercise intensity is increasing and entropy is decreasing, this indicates that the AT point has not been reached as discussed above, in which case it is necessary to continue to increase exercise intensity and calculate PI and entropy values. If the AT has not been reached, then in step Sc9, the microprocessor 117 determines if the current workout stage has ended. If the current workout stage has not ended, then the flow returns to step Sc5. However, if the current workout stage has ended, then the exercise intensity is increased in step Sc10, after which the flow returns to step Sc5.

When entropy is at a minimum, then the AT point can be obtained in step Sc8. That is, if the AT point has been reached in step Sc8, then step Sc11 is performed, in which the microprocessor 117 performs control to display, record, and store the obtained AT and exercise data of the user performing exercise, after which the process is ended.

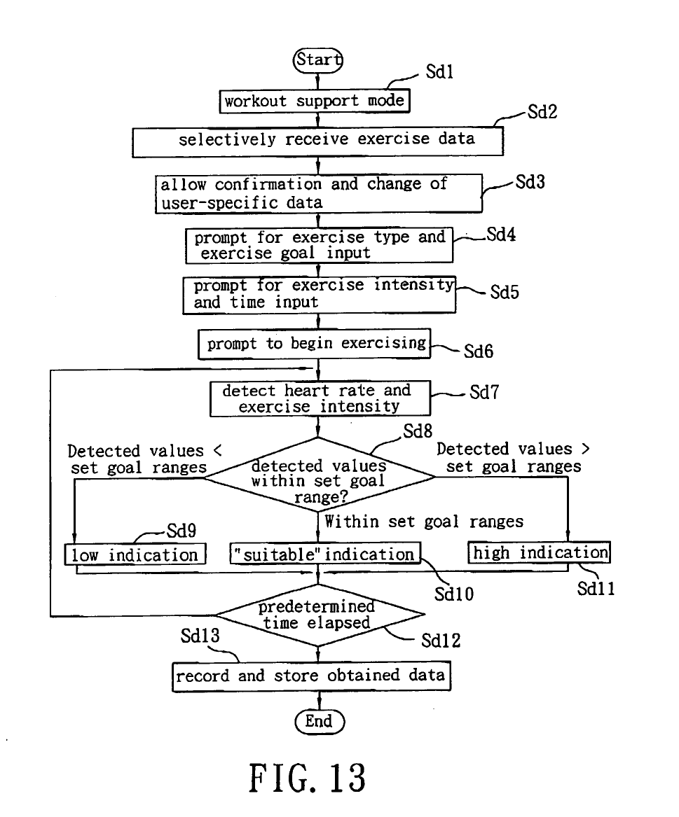

FIG. 13 shows an example of processes involved in a workout support function of the mobile phone apparatus 1 of the present invention.

First, in step Sd1, the user manipulates the user interface 108 to place the mobile phone apparatus 1 in a workout support mode. Next, in step Sd2, the microprocessor 117 performs control to selectively receive exercise data. In step Sd3, the user is prompted by control of the microprocessor 117 to check and change (if necessary) user-specific data, such as height, weight, sex, and age. As an example, the microprocessor 117 may perform control to prompt the user via the display unit 117, and the user may then manipulate the user interface 108 to perform the required input.

Subsequently, in step Sd4, the user is prompted by control of the microprocessor 117 to select a particular exercise and an exercise goal. For example, the user may select one of jogging, walking, and cycling as the exercise he or she intends to perform, and may select one of cardio-respiratory fitness and weight reduction as the exercise goal. Next, in step Sd5, the user is prompted by control of the microprocessor 117 to select exercise intensity and exercise time. The exercise intensity may be established based on the previously to measured and stored \dot{V}O₂max and AT, or may be determined based on program instructions stored in the ROM 113. As an example of the former method, when the user has selected a weight reduction exercise goal, the exercise intensity may be set at 80% of AT.

Next, in step Sd6, the microprocessor 117 performs control to prompt the user to begin exercising and increase exercise intensity as needed. This may include a prompt for the user to first perform a warm-up stage of exercise, after which the user performing exercise is prompted to increase exercise intensity as needed.

Next, in step Sd7, the detecting assembly 12 of the mobile phone apparatus 1 detects heart rate and exercise intensity. After this step, the microprocessor 117 compares the detected values with predetermined values instep Sd8 to determine if the detected values are within Bet goal ranges. If the detected values are less than the goal ranges, then the microprocessor 117 performs control to provide indication to the user performing exercise in step Sd9 that the exercise intensity is too low. If the detected values exceed the goal ranges, then the microprocessor 117 performs control to provide an indication to the user performing exercise in step Sd11 that the exercise intensity is too high. Finally, if the detected values fall within the set goal ranges, then a “suitable” indication is provided to the user performing exercise in step Sd10. The user performing to exercise may adjust his or her exercise intensity as needed.

After any of the steps Sd9, Sd10, and Sd11, the microprocessor 117 determines if a predetermined time has elapsed in step Sd12. If the predetermined time has not elapsed, then the flow returns to step Sd7. However, if the predetermined time has elapsed, then the microprocessor 117 performs control to record and store the data obtained during exercise in the RAM 114.

In the present invention, regardless of whether VO2max or AT is estimated and of the method used in measuring VO2max or AT, exercise intensity must be progressively increased, and physiological parameters (such as heart rate or pulse rate) must be monitored to ensure that they are within safety ranges. While the safety limit in the preferred embodiment is HRL=0.85 (220−age), the present invention should not be limited thereto. In other embodiments, a maximal heart rate (Hrmax) equal to (220−age) can be applied as a heart rate limit indicative of the condition that the exercise load has reached a maximal value. Furthermore, it is also possible to reach a conclusion that the AT point has been reached with reference to the heart rate. That is, HR(AT)=0.55(220−age).

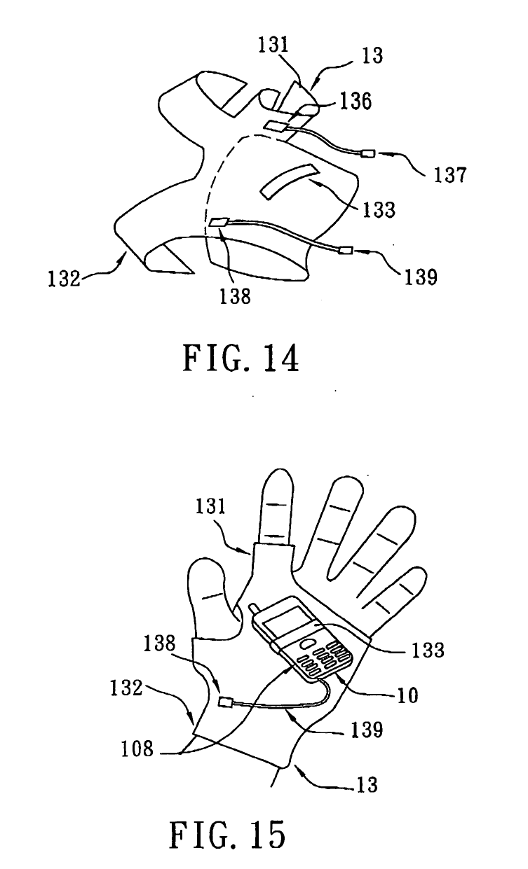

Referring to FIGS. 14, 15, and 16, a holder 13 may be used to secure the mobile phone apparatus 1 of the present invention on the user performing exercise. It will be assumed for the following discussion that the mobile phone apparatus 1 is configured as the mobile phone shown in FIG. 3. The holder 13 allows for real-time remote monitoring, and fully secures the mobile phone apparatus 1 so that the user may perform exercise without the mobile phone apparatus 1 being removed from the holder 13. The holder 13 may also serve as an external detector for the mobile phone apparatus 1 (i.e., a pulse detector).

To perform these functions, the holder 13 must satisfy a plurality of conditions (assuming once again that the mobile phone apparatus 1 is configured as a mobile phone) First, the holder 13 must be able to firmly secure the mobile phone apparatus 1 to the body of the user performing exercise such that the motion detector 121 of the detecting assembly 12 is able to accurately detect movement of the user's body. Second, the holder 13 must be able to conveniently detect physiological parameters, such as pulse rate. Third, the holder 13 must allow for convenient access to the mobile phone apparatus 1 so that the user may easily manipulate the user interface 108. Finally, the holder 13 must allow the user to easily view or sense signals output by the mobile phone apparatus 1, such as display signals, audio signals, and vibration alert signals.

The holder 13 according to a preferred embodiment of the present invention includes a securing strap 133, first and second fastening belts 131,132, a detecting unit including first and second detecting elements 136, 138, and first and second transmission lines 137, 139 to facilitate coupling between the detecting unit of the holder 13 and the mobile phone apparatus 1. The strap 133 is used to secure the mobile phone apparatus 1 to the holder 13. The second fastening belt 132 is used to secure the holder 13 to the wrist of the user. The second detecting element 138 is positioned at an inner surface of the second fastening belt 132, and may be configured as a piezoelectric microphone to detect the pulse of the user performing exercise The detected pulse signals are received by the mobile phone apparatus 1 through the second transmission line 139. In addition, the first detecting element 136 is positioned at an inner surface of the first fastening belt 131, and may be configured as an optical pulse reader which measures movement of blood in the capillaries of the finger to thereby generate pulse signals that may be used to calculate pulse rate, and that are provided to the mobile phone apparatus 1 through the first transmission line 137.

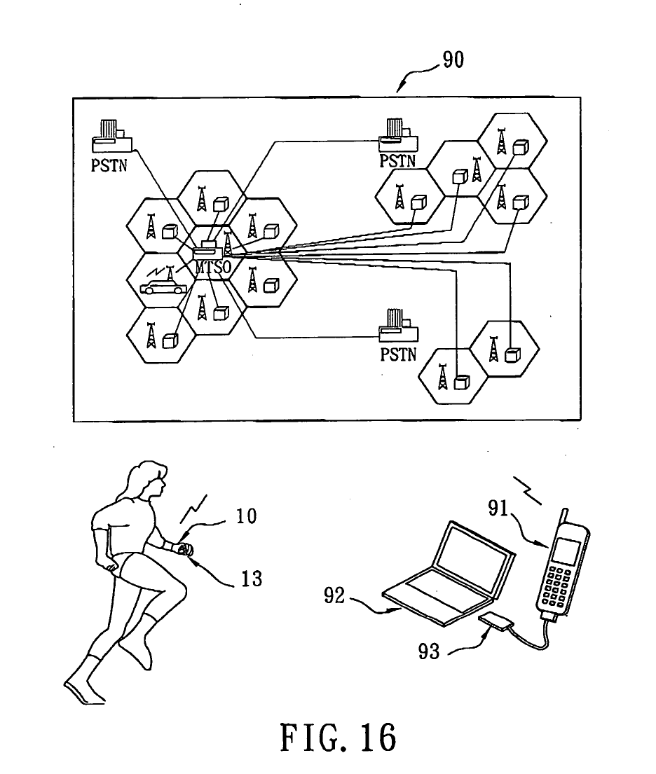

Referring to FIG. 16, the user may set up the mobile phone apparatus 1 such that exercise data obtained during exercise are transmitted to another mobile communication device 91 via a mobile phone network 90. The other mobile communication device 91 maybe connected to a personal computer (PC) 92 in a known manner to thereby allow for processing of the exercise data by the personal computer 92. The personal computer 92 may also send instructions back to the mobile phone apparatus 1 in the same manner, thereby realizing real-time remote monitoring and control.

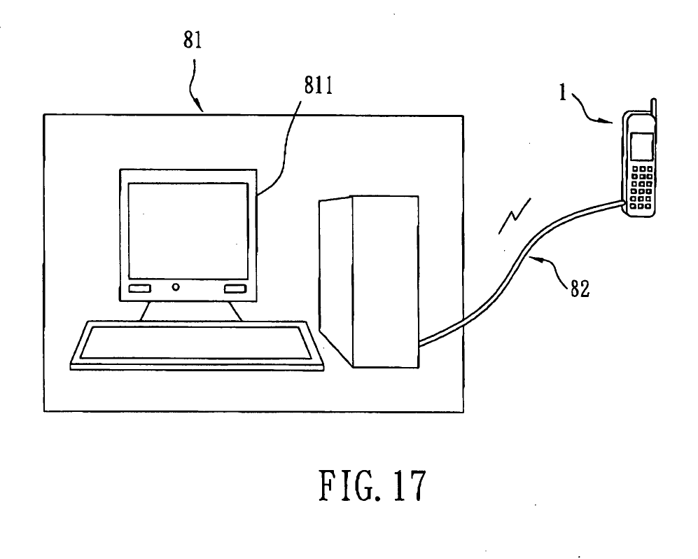

Referring to FIG. 17, the mobile phone apparatus 1 maybe used to transmit data signals to and from a personal computer 81. Following completion of exercise, the mobile phone apparatus 1 may transmit, either through a wire 82 or by wireless connection, the exercise data stored in the mobile phone apparatus 1 to the personal computer 81. The higher computational capability of the personal computer 81 may then be used to process the data, and the results of such processing may then be displayed on a display 811 of the personal computer 81. The data may also be stored in the personal computer 81 for future reference or for comparison with other exercise data so that other workout training programs may be designed accordingly.

In the mobile phone apparatus 1 of the present invention described above, sports physiological measurements of the user performing exercise are detected. The obtained data may then be used to estimate \dot{V}O₂max and AT. These exercise performance indicia may then, in turn, be used to provide workout support through video, audio and/or vibration alert interaction with the user. Hence, the user performing exercise may easily and effectively obtain highly useful information regarding his or her state of physical fitness, and may be aided during his or her exercise regimen to perform exercise in a safe and effective manner. In sum, the present invention basically utilizes a mobile phone apparatus 1 including three detectors 121, 122, 123 to generate two biological indexes during exercise. Therefore, there are at least four marked distinctions between the present invention and U.S. Pat. No. 6,817,979.

First, the biological sports physiology indexes, i.e., VO2max and AT, have clear and strict definitions in sports physiology. The aforesaid U.S. Pat. No. 6,817,979 is totally silent on this aspect.

Second, the present invention simulates and replaces the control functions of costly exercising apparatuses (such as bicycle ergometers, treadmills, step exercisers, etc.) in laboratories or health clubs that can be precision-set to progressively increase exercise intensities (fixed time intervals and fixed amount).

Third, the present invention employs computational software to further perform integration, computation and determination of the heart rate, physical fitness data, and exercise intensities so as to obtain the two biological sports physiology indexes disclosed in this invention.

Using the computational software, the present invention can then apply the two biological sports physiology indexes to make exercising load settings, perform exercises, and monitor exercise performance.

In addition, the mobile phone apparatus 1 of the present invention may be configured for data processing using solely the computational software stored in the ROM 113, and does not need to process data through a network server as taught in the aforesaid U.S. Pat. No. 6,817,979.

Finally, through use of the configuration of the present invention described above, i.e., the configuration providing the mobile phone apparatus 1 with exercise measuring and workout support capabilities, and through use of the inherent wireless transmission capabilities of the mobile phone apparatus 1, real-time monitoring and control during exercise is made possible.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A mobile phone apparatus for performing sports physiological measurements and generating target workout information, comprising:
a motion detector for detecting motion of a user performing exercise;
a physiological parameter detector adapted to be placed in contact with the body of the user performing exercise, said physiological parameter detector detecting at least one physiological parameter of the user performing exercise;
a portable housing, said physiological parameter detector being mounted at least partially external to said portable housing; and
a processing module mounted in said portable housing and coupled to said motion detector and said physiological parameter detector, said processing module including
a performance estimating monitor for estimating at least one of a maximum oxygen uptake quantity (\dot{V}O₂max) and an anaerobic threshold (AT) of the user performing exercise with reference to data obtained by said motion detector and said physiological parameter detector, a workout training program for establishing a series of workout stages having varying exercise intensities to be targeted by the user performing exercise, and
a target performance indicator for generating target workout information to the user performing exercise with reference to data obtained by said motion detector and said physiological parameter detector, as well as said workout training program.

2. The mobile phone apparatus of claim 1, wherein said processing module includes a processor and a program memory coupled to said processor, said program memory storing program instructions executed by said processor for configuring said processing module to include said workout training program, said performance estimating monitor, and said target performance indicator.

3. The mobile phone apparatus of claim 1, further comprising a display unit coupled to said processing module and operable so as to show the target workout information thereon.

4. The mobile phone apparatus of claim 1, further comprising at least one of a vibration alert unit and a buzzer unit coupled to said processing module and operable so as to provide an alert signal corresponding to the target workout information.

5. The mobile phone apparatus of claim 1, wherein said motion detector and said physiological parameter detector are disposed externally of said portable housing.

6. The mobile phone apparatus of claim 1, wherein said motion detector includes an accelerometer associated operably with said processing module to enable said performance estimating monitor to determine exercise intensity of the user performing exercise.

7. The mobile phone apparatus of claim 6, wherein said performance estimating monitor determines at least one of number of paces per unit time, motion speed, distance traveled, and work exerted by the user performing exercise in power units with reference to data obtained by said accelerometer.

8. The mobile phone apparatus of claim 1, wherein said physiological parameter detector is adapted to detect at least one of a heart rate and a pulse rate of the user performing exercise.

9. The mobile phone apparatus of claim 1, further comprising an environment detector coupled to said processing module and operable so as to detect environmental conditions to obtain environmental data, said performance estimating monitor suitably factoring in the environmental data when estimating the \dot{V}O₂max and the AT of the user performing exercise.

10. The mobile phone apparatus of claim 1, further comprising a subscriber identity module card mounted in said portable housing and coupled to said processing module.

11. The mobile phone apparatus of claim 1, further comprising a user interface coupled to said processing module and operable so as to input user-specific data, said performance estimating monitor suitably factoring in the user-specific data when estimating the \dot{V}O₂max and the AT of the user performing exercise.

12. The mobile phone apparatus of claim 11, wherein said user interface includes a keypad mounted on said portable housing.

13. The mobile phone apparatus of claim 11, wherein the user-specific data includes at least one of height, weight, age, and sex of the user performing exercise.

14. The mobile phone apparatus of claim 13, wherein said performance estimating monitor determines at least one of number of paces per unit time, motion speed, distance traveled, and work exerted by the user performing exercise in power units with reference to data obtained by said motion detector, and further calculates exercise intensity as a function of the number of paces per unit time, a stride distance parameter, and the weight of the user performing exercise.

15. The mobile phone apparatus of claim 14, further comprising an environment detector coupled to said processing module and operable so as to detect environmental conditions to obtain environmental data, said performance estimating monitor estimating the \dot{V}O₂max from: (a) at least three sets of the data from said physiological parameter detector and the calculated exercise intensities associated respectively therewith, said at least three sets having a linear relationship; (b) the sex, age and weight of the user performing exercise; and (c) the environmental data.

16. The mobile phone apparatus of claim 14, wherein said performance estimating monitor estimates entropy for different exercise intensities, said performance estimating monitor associating the AT with the smallest entropy estimated thereby.

17. The mobile phone apparatus of claim 14, wherein said performance estimating monitor estimates power of heart rate variability for different exercise intensities, said performance estimating monitor associating the AT with the power of heart rate variability estimated thereby.

18. The mobile phone apparatus of claim 1, wherein the target workout information generated by said target performance indicator is for indicating to the user performing exercise as to times when the exercise intensities to be targeted by the user are to be progressively increased starting from a warm-up stage in accordance with said workout training program.

19. The mobile phone apparatus of claim 18, wherein said target performance indicator alerts the user performing exercise if the data obtained by said physiological parameter detector is not within a safety range, and enables progressive increase in the exercise intensity between successive workout stages with reference to said at least one of the \dot{V}O₂max and the AT estimated by said performance estimating monitor.

20. The mobile phone apparatus of claim 1, further comprising a holder for securing said portable housing to the user performing exercise, said holder including a securing strap for securing said portable housing to said holder, and a fastening belt adapted to secure said holder to a body part of the user performing exercise,
at least one of said motion detector and said physiological parameter detector being mounted on said holder.

21. The mobile phone apparatus of claim 20, wherein said holder further includes a transmission line unit for coupling said processing module to said at least one of said motion detector and said physiological parameter detector mounted on said holder.

22. The mobile phone apparatus of claim 1, wherein said performance estimating monitor estimates AT of the user performing exercise with reference to data obtained by said motion detector and said physiological parameter detector using a regression analysis method.

23. The mobile phone apparatus of claim 22, wherein the regression analysis method comprises a linear regression method.

24. The mobile phone apparatus of claim 22, wherein the regression analysis method comprises a third-order curvilinear regression method.

25. The mobile phone apparatus of claim 22, wherein the regression analysis method comprises a logistical growth function method.
